(19) United States
(12) Patent Application Publication
(10) Pub. No.: US 2010/0331409 A1
(43) Pub. Date: Dec. 30, 2010

(54) Use of Methyl Cinnamate in the Inhibition of Candida Albicans Biofilms

(75) Inventors: Ying-Chieh Tsai, Taipei (TW); Meng-Hwan Lee, Yunlin County (TW); Chih-Chieh Hsu, Taipei (TW); Kuei-Chin Chuang, Taoyuan County (TW)

Correspondence Address: WPAT, PC;INTELLECTUAL PROPERTY ATTORNEYS, 2030 MAIN STREET, SUITE 1300, IRVINE, CA, 92614, US

(73) Assignee: Barak Biotechnology Co., Ltd., Taipei (TW)

(21) Appl. No.: 12/821,497

(22) Filed: Jun. 23, 2010

(30) Foreign Application Priority Data
Jun. 24, 2009 (TW) 098121253

(51) Int. Cl. A61K 31/235; A61P 31/00

(52) U.S. Cl. 514/532

(57) ABSTRACT

The invention provides a method of inhibiting biofilm formation or development of Candida albicans, comprising applying to an object in need thereof an effective amount of methyl cinnamate whereby formation or development of Candida albicans biofilm can be inhibited or prevented. Also provided is a method of treating Candida albicans biofilm-associated infections, comprising administrating to a subject in need thereof a therapeutically effective amount of methyl cinnamate.

### FIELD OF THE INVENTION

The invention provides a method of inhibiting biofilm formation or development of Candida albicans, comprising applying to an object in need thereof an effective amount of methyl cinnamate whereby formation or development of Candida albicans biofilm can be inhibited or prevented. Also provided is a method of treating Candida albicans biofilm-associated infections, comprising administrating to a subject in need thereof a therapeutically effective amount of methyl cinnamate.

### BACKGROUND OF THE INVENTION

Biofilms possess unique developmental characteristics that are in stark contrast to the characteristics of free-floating cells, and biofilms are much more difficult to treat chemotherapeutically. A biofilm is a structured community of microorganisms encapsulated within a self-developed polymeric matrix and adherent to a living or inert surface. Biofilms are also often characterized by surface attachment, structural heterogeneity, genetic diversity, complex community interactions, and an extracellular matrix of polymeric substances. Formation of a biofilm begins with the attachment of free-floating microorganisms to a surface. These first colonists adhere to the surface initially through weak, reversible van der Waals forces. If the colonists are not immediately separated from the surface, they can anchor themselves more permanently using cell adhesion structures such as pili. The first colonists facilitate the arrival of other cells by providing more diverse adhesion sites and beginning to build the matrix that holds the biofilm together. Only some species are able to attach to a surface on their own. Others are often able to anchor themselves to the matrix or directly to earlier colonists. Once colonization has begun, the biofilm grows through a combination of cell division and recruitment.

Biofilms occur in medical equipment, such as catheters, and are a major source of hospital infections. Biofilms can also occur in areas such as contact lenses, other medical equipment, and other industries. A primary difficulty with biofilms is that they are more difficult to reduce or eliminate than are individual bacteria. This is due to the formation of the protective layer of slime, as well as adaptations that the individual bacteria undergo when they form biofilms. Microorganisms living in a biofilm can have significantly different properties from free-floating microorganisms, as the dense and protected environment of the film allows them to cooperate and interact in various ways. One benefit of this environment is increased resistance to detergents and antibiotics, as the dense extracellular matrix and the outer layer of cells protect the interior of the community.

The yeast Candida albicans is a commensal of human mucosal surfaces and also an opportunistic pathogen. Candida albicans causes a wide variety of diseases including oral thrush and disseminated candidiasis. Systemic fungal infections have emerged as important causes of morbidity and mortality in immunocompromised patients (e.g., as a result of AIDS, cancer chemotherapy, organ or bone marrow transplantation). In addition, hospital-related infections in patients not previously considered at risk (e.g., patients in an intensive care unit) have become a cause of major health concern. Candida albicans is also the major fungus that colonizes medical implants, causing device-associated infections with high mortality. (Kojic E M, Darouiche R O: Candida Infections of Medical Devices. Clin Microbiol Rev 2004, 17:255-267; Nobile et al., Critical Role of Bcr1-dependent Adhesins in Candida albicans Biofilm Formation In Vitro and In Vivo. PLoS Pathog. 2006, 2: e63). Infections involving medical devices are notoriously difficult to eliminate and generally necessitate removal of the device. Candida albicans colonizes the surfaces of catheters, prostheses, and epithelia, forming biofilms that are extremely resistant to antifungal drugs. Mature Candida albicans biofilms show a complex three-dimensional architecture with extensive spatial heterogeneity, and consist of a dense network of yeast, hyphae and pseudohyphae encased within a matrix of exopolymeric material. Donlan RM reported that the statistic data of National Institutes of Health, USA (NIH) in 2000 indicated that 80% microbial infection cases were caused by biofilms; for example, cystic fibrosis, native valve endocarditis, otitis media, periodontitis and chronic prostatitis (Donlan R M, Biofilms: Microbial Life on Surfaces, Emerg Infect Dis 2002, 8:881-890). Moreover, the medical devices contaminated by microorganisms are main cause of infection in hospitals wherein around 50% infection are caused by biofilms formed on medical devices (Kojic E M, Darouiche R O, Candida Infections of Medical Devices, Clin Microbiol Rev 2004, 17:255-267). Biofilms of Candida albicans are less susceptible to many antifungal drugs than are planktonic cells. Antifungal drug resistance in Candida albicans biofilms seems to be the result of several factors. It has been reported that cells in biofilms have a reduced content of ergosterol compared with cells in liquid culture. In addition, cells in biofilms express genes encoding drug efflux determinants including CDR1 and CDR2 and transient expression of MDR1 has also been observed in adherent cells. All of these factors could contribute to the high levels of drug resistance exhibited by Candida albicans cells in a biofilm. The azoles, such as fluconazole, are one major class of effective antifungals; they act through inhibition of ergosterol biosynthesis. Candida albicans azole resistance has been known for some time in the clinic, and several resistance mechanisms are well characterized. (Pranab K. Mukherjee et al., Mechanism of Fluconazole Resistance in Candida albicans Biofilms: Phase-Specific Role of Efflux Pumps and Membrane Sterols, Infect Immun, 2003, 71: 4333-4340). LaFleur M D et al. reported that biofilms formed by the major human pathogen Candida albicans exhibited a strikingly biphasic killing pattern in response to two microbicidal agents, amphotericin B, a polyene antifungal, and chlorhexidine, an antiseptic, indicating that a subpopulation of highly tolerant cells, termed persisters, existed. (LaFleur M D et al., Candida albicans Biofilms Produce Antifungal-tolerant Persister Cells, Antimicrob Agents Chemother, 2006 50:3839-46.)

Several studies have shown uniform resistance of the organisms in the biofilm to a wide spectrum of conventional antifungal agents including resistance to the new triazoles (VRC and Ravu), which have been shown to be fungicidal with extended activity against many azole-resistant organisms. Therefore, biofilm-associated infections are difficult to treat, which emphasizes the need to develop antimicrobial drugs that show activity against biofilm-associated organisms and specifically target biofilm-associated infections.

### SUMMARY OF THE INVENTION

The invention provides a method of inhibiting or preventing biofilm formation of Candida albicans, comprising applying to an object in need thereof an effective amount of methyl cinnamate whereby formation of Candida albicans biofilms can be inhibited or prevented.

The invention also provides a method of treating Candida albicans biofilm-associated infections, comprising administrating to a subject in need thereof a therapeutically effective amount of methyl cinnamate.

### BRIEF DESCRIPTION OF THE DRAWING

FIG. 1 shows the development biofilm of Candida albicans on polystyrene material, wherein T represents time and the scale bar is 30 μm.

FIG. 2 shows that the development of biofilm on polystyrene reached a steady state and the values of XTT and CV remained at a range. Candida albicans was cultured at 37° C. After 1 hour, the cells of Candida albicans adhered to the surface of polystyrene material (T=0). The cells were continuously cultured at 37° C. and the amounts of the biofilm were determined by XTT and crystal violet at different time points.

FIG. 3 shows the inhibition of biofilm of Candida albicans with methyl cinnamate. FIGS. 3(A) and 3(B) represent the results determined by XTT and crystal violet.

FIG. 4 shows the inhibition of biofilm of Candida albicans with the essential oil of Zanthoxylum armatum. FIGS. 4(A) and 4(B) represent the results determined by XTT and crystal violet.

### DETAILED DESCRIPTION OF THE INVENTION

The invention discovers a new use of methyl cinnamate in the inhibition or prevention of biofilm formation of Candida albicans. The invention confirms that methyl cinnamate is a molecule with ability to inhibit or prevent the biofilm formation of Candida albicans cells. By inhibiting or preventing biofilm formation of Candida albicans, methyl cinnamate can be used to inhibit or prevent development of Candida albicans in an object. In addition, methyl cinnamate can disrupt the structure of the biofilm, so it can be used to treat biofilm-associated infections caused by Candida albicans.

Hence, the invention provides a method of inhibiting biofilm formation of Candida albicans, comprising applying to an object on which Candida albicans may form biofilms an effective amount of methyl cinnamate whereby formation of Candida albicans biofilms can be inhibited or prevented. Also, the invention provides a method of treating Candida albicans biofilm-associated infections, comprising administrating to a subject in need thereof a therapeutically effective amount of methyl cinnamate.

Methyl cinnamate is the methyl ester of cinnamic acid and has the structure below:

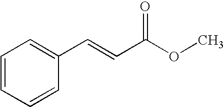

It is found naturally in a variety of plants, including in fruits, such as strawberry, and some culinary spices, such as Sichuan pepper, some varieties of basil and Zanthoxylum armatum. Methyl cinnamate is used in the flavor industry and in perfumes to impart strawberry and cinnamon scents. The invention finds that methyl cinnamate can inhibit the hyphal formation of Candida albicans and disrupt its biofilm structure so that it can be used as an agent against Candida albicans biofilms and to treat Candida albicans biofilm-associated infections. Hence, the invention also provides a method of preventing or inhibiting formation of Candida albicans biofilm, comprising applying to an object in need thereof an effective amount of essential oils of Zanthoxylum armatum. Furthermore, the invention found that the essential oil of Zanthoxylum armatum contains large amount of methyl cinnamate, so it also can inhibit or prevent biofilm formation of Candida albicans. A method of treating Candida albicans biofilm-associated infections, comprising administrating to a subject in need thereof a therapeutically effective amount of essential oils of Zanthoxylum armatum, is also provided.

The term “therapeutically effective amount” for administrating to a subject, as used herein, refers to the amount of an agent that, when administered to a subject, is effective to at least partially prevent and/or cure (or prevent and/or inhibit) an infection condition caused by Candida albicans from which the subject is suspected to suffer (or an undesired condition). The term “effective amount” for applying to an object, as used herein, refers to the amount of an agent that, when applied to an object, is effective to at partially prevent and/or inhibit the development of biofilm of Candida albicans.

The term “subject,” as used herein, refers to a mammal, preferably a human.

The term “object,” as used herein, refers to any object on which Candida albicans biofilm may form. Preferably, the object is selected from the group consisting of plastics, polymeric materials, medical implants, medical devices, prosthesis and catheters.

The terms “administer,” “administering” or “administration,” as used herein, refer to either directly administering a compound or composition to a patient, or administering a derivative or analog of the compound to the patient, which will form an equivalent amount of the active compound or substance within the patient's body.

The terms “treat,” “treating” or “treatment,” as used herein, include alleviating, abating or ameliorating the symptoms of a disease or condition, preventing additional symptoms, or inhibiting the disease or condition, e.g., arresting the development of the disease or condition, relieving the disease or condition, causing regression of the disease or condition, relieving a condition caused by the disease or condition, or stopping the symptoms of the disease or condition either prophylactically and/or therapeutically.

The terms “prevent,” “preventing” or “prevention,” as used herein, refers to the administration or application of methyl cinnamate or its derivatives of the present invention to reduce or avoid the biofilm formation of Candida albicans.

Any suitable route of administration or application can be employed to provide the subject or object with an (therapeutically) effective amount of methyl cinnamate of the present invention. For administration, oral, parenteral (i.e. subcutaneous, intramuscular, intravenous, etc.), transdermal, aerosol and other forms of administration can be employed. Dosage forms include, but are not limit to, tablets, troches, dispersions, suspensions, solutions, capsules, suppositories, microencapsulated systems, slow-release and controlled-release systems, transdermal delivery systems, including, for example patches, creams, ointments and electrophoretic systems, and the like. For application, the applying forms include, but are not limit to, coatings, aerosols, solutions, emulsions, pastes, gels and powders.

According to the invention, the application amount of methyl cinnamate used in the object of the method of the invention ranges from 0.015% to 0.8%. A preferred amount range is 0.015% to 0.125%. Other preferred amount ranges include 0.125% to 0.8%. On the other hand, the amount of essential oil of Zanthocylum armatum used in the method of the invention ranges from 0.008% to 0.3%. A preferred amount range is 0.008% to 0.125%. Other preferred amount ranges include 0.125% to 0.3%.

According to the invention, the administration dosage of methyl cinnamate used in the subject of the method of the invention ranges from 0.015% to 0.8%. A preferred dose range is 0.015% to 0.125% . Other preferred dose ranges include 0.125% to 0.8%. On the other hand, the dosage of essential oil of Zanthoxylum armatum used in the method of the invention ranges from 0.008% to 0.3%. A preferred dose range is 0.008% to 0.125% . Other preferred dose ranges include 0.125% to 0.3%.

The invention finds that the concentrations of methyl cinnamate and essential oil of Zanthoxylum armatum against biofilm of Candida albicans are much lower than their minimum inhibition concentration and minimum fungicidal concentration. It shows that methyl cinnamate and essential oil of Zanthoxylum armatum indeed inhibit formation and development of biofilm of Candida albicans rather than inhibit or kill Candida albicans per se.

### Examples

### Example 1

### Development of Biofilms of Candida albicans

A single colony of Candida albicans was selected from YPD plate (1% (w/v) yeast extract, 2% (w/v) peptone, 2% (w/v) dextrose and 1.5% (w/v) agar), seeded in YPD liquid medium (1% (w/v) yeast extract, 2% (w/v) peptone and 2% (w/v) dextrose) and then cultured at 30° C. and 100 rpm for 16 hours. The YPD medium was washed out and the amounts of the Candida albicans cells were counted using Hematocyte Counter. The cells were diluted with RPMI 1640 medium (Gibco, Invitroge, Calif., U.S.A.) to 5×10⁵ cells/ml. 200 μL of 5×10⁵ cells/ml medium were added to 96-well plate and placed at 37° C. for 1 hour so that the cells could adhere to the bottom of the plate. The non-adhered cells were washed out with phosphate buffer. The material of the 96-well plate was polystyrene, which is a common material for medical device such as syringe. Subsequently, RPMI 1640 medium was added to the plate and cultured at 37° C. To remove the planketic cells, PBS was added to wash them at different time points. The numbers of cells in the biofilm were determined by a colorimetric assay using XTT [2,3-Bis(2-methoxy-4-nitro-5-sulfo-phenyl)-2H-tetrazolium-5-carboxanilide] (Sigma, Mo., U.S.A.) and their biomass was determined by crystal violet to find out the mature development of biofilm (Ramage G et al., Inhibition of Candida albicans Biofilm Formation by Farnesol, A Quorum-Sensing Molecule. Appl Environ Microbiol 2002, 68: 5459-5463). Six hours after the cells adhered to the polystyrene plate, hyphae or pseudohyphae would grow. After further culture at 37° C. for 6 hours, yeast-like Candida albicans appeared and gradually secreted extracellular materials. Subsequently, hyphae- and yeast-like Candida albicans developed simultaneously and their numbers and distribution increased rapidly. Candida albicans tends to aggregate and develop colonies. After culture at 37° C. for 24 hours, the formation of biofilm can be observed. The development of biofilm of Candida albicans on polystyrene had the following three stages:

(1) Initial development stage: The time for this stage was 0 to 6 hours and no yeast-like cells could be observed.

(2) Medium development stage: The time for this stage was 6 to 24 hours and yeast-like cells appeared and increased. Hyphae- and yeast-like cells massively increased and colonies formed.

(3) Last development stage: the time for this stage was more than 24 hours. The biofilm could be observed at the bottom of the plate.

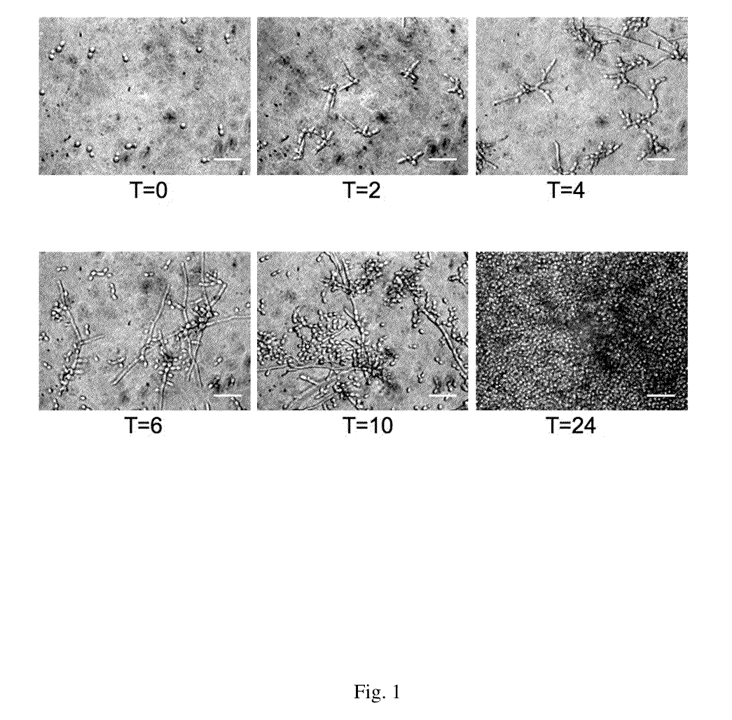

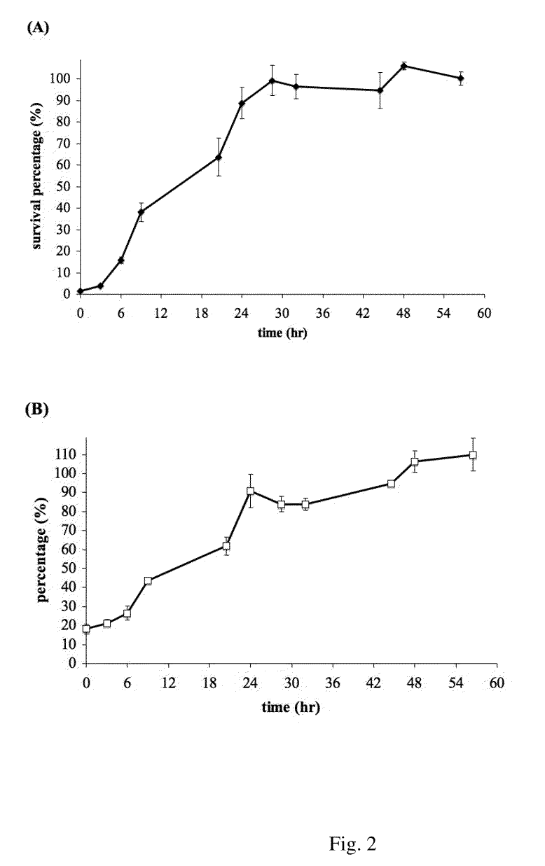

The above-mentioned development of biofilm of Candida albicans is shown in FIG. 1. After colorimetric assays using XTT and crystal violet (CV), it was observed that the development of biofilm on polystyrene reached a steady state and the values of XTT and CV remained at a range (see FIG. 2). Therefore, the maturation of biofilm development of Candida albicans on polystyrene is at around 24 hours.

### Example 2

### Minimum Inhibition Concentrations (MIC) and Minimum Fungicidal Concentrations (MFC) of Methyl Cinnamate and Essential Oil of Zanthoxylum armatum Against Candida albicans

### MIC Assay

A single colony of Candida albicans was selected from YPD plate, seeded in YPD liquid medium and then cultured at 30° C. and 100 rpm for 16 hours. The medium was diluted to 1×10⁵ CFU/ml. The diluted medium was added to 96-well plates containing different concentrations of methyl cinnamate and essential oil of Zanthoxylum armatum (0.00625% to 1% for methyl cinnamate and 0.025% to 0.5% for essential oil) and cultured at 37° C. for 16 hours. It was found that the MIC of methyl cinnamate was 0.1% and that of essential oil of Zanthoxylum armatum was 0.15%.

### MFC Assay

A single colony of Candida albicans was selected from YPD plate, seeded in YPD liquid medium and then cultured at 30° C. and 100 rpm for 16 hours. The medium was diluted to 1×10⁵ CFU/ml. The diluted medium was added to 96-well plates containing different concentrations of methyl cinnamate and essential oil of Zanthoxylum armatum (0.00625% to 1% for methyl cinnamate and 0.025% to 0.5% for essential oil) and cultured at 37° C. for 16 hours. It was found that the MFC of methyl cinnamate was 0.8% and that of essential oil of Zanthoxylum armatum was 0.3%.

### Example 3

### Inhibition of Biofilm of Candida albicans with Methyl Cinnamate and Essential Oil of Zanthoxylum Armatum

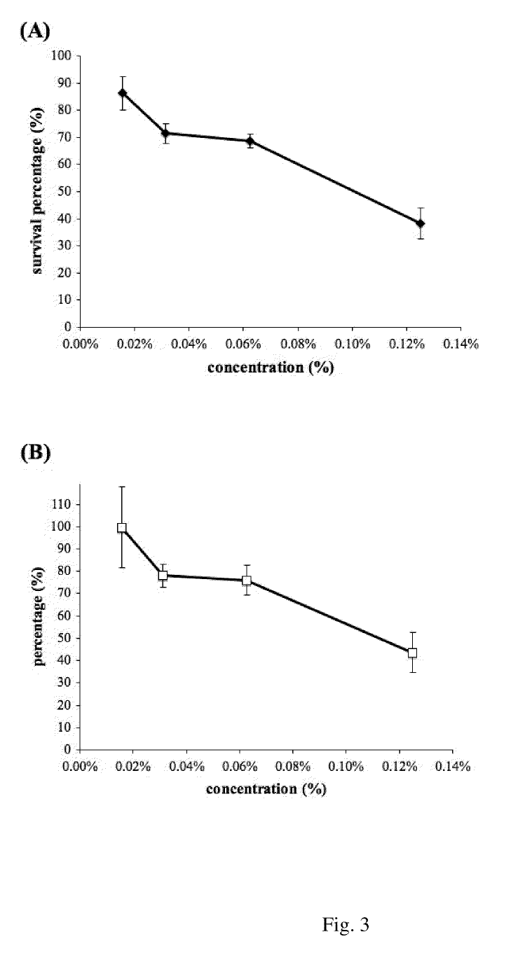

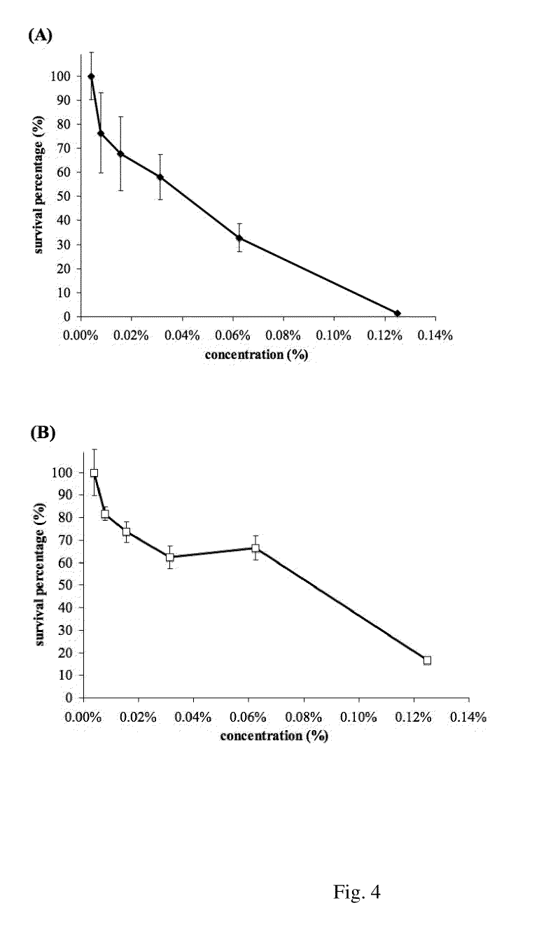

After the cells of Candida albicans were cultured at 37° C. for 1 hour, they adhered to the 96-well plate made with polystyrene material. After continuous culture at 37° C. for 4 hours, various concentrations of methyl cinnamate and essential oil of Zanthoxylum armatum were added to the plate and cultured with the cells at 37° C. to 48 hours, and the numbers of the viable cells and their biomass were determined by XTT (FIGS. 3a and 4a) and CV (FIGS. 3b and 4b) assays, respectively. 1% methanol was used as control. FIGS. 3 and 4 show the inhibition of biofilm of Candida albicans with methyl cinnamate and essential oil of Zanthoxylum armatum, respectively. 10 to 60% of biofilm was inhibited by the concentration ranging from 0.015% to 0.125% of methyl cinnamate. Around 50% and 70% of biofilm can be inhibited by 0.03% and 0.06% essential oil of Zanthoxylum armatum, respectively. The above concentrations of methyl cinnamate and essential oil of Zanthoxylum armatum are much lower than their MICs and MFCs.

It shows that methyl cinnamte and essential oil of Zanthoxylum armatum indeed inhibit formation and development of biofilm of Candida albicans rather than inhibit or kill Candida albicans per se.

What is claimed is:

1. A method of inhibiting or preventing biofilm formation of Candida albicans, comprising applying to an object in need thereof an effective amount of methyl cinnamate whereby formation of Candida albicans biofilms can be inhibited or prevented.

2. The method of claim 1, wherein the object is selected from the group consisting of plastics, polymeric materials, medical implants, medical devices, prosthesis and catheters.

3. The method of claim 1, wherein the amount of methyl cinnamate ranges from 0.015% to 0.8%.

4. The method of claim 1, wherein the amount of methyl cinnamate ranges from 0.015% to 0.125%.

5. The method of claim 1, wherein the amount of methyl cinnamate ranges from 0.125% to 0.8%.

6. The method of claim 1, wherein methyl cinnamate is applied in the form of coatings, aerosols, solutions, emulsions, pastes, gels or powders.

7. The method of claim 1, wherein the methyl cinnamate is contained in essential oil of Zanthoxylum armatum.

8. The method of claim 7, wherein the amount of essential oil of Zanthoxylum armatum ranges from 0.008% to 0.3%.

9. The method of claim 7, wherein the amount of essential oil of Zanthoxylum armatum ranges from 0.008% to 0.125%.

10. The method of claim 7, wherein the amount of essential oil of Zanthoxylum armatum ranges from 0.125% to 0.3%.

11. A method of treating Candida albicans biofilm-associated infections, comprising administrating to a subject in need thereof a therapeutically effective amount of methyl cinnamate.

12. The method of claim 11, wherein the subject is a mammal.

13. The method of claim 11, wherein the subject is a human.

14. The method of claim 11, wherein the dosage of methyl cinnamate ranges from 0.015% to 0.8%.

15. The method of claim 11, wherein the dosage of methyl cinnamate ranges from 0.015% to 0.125%.

16. The method of claim 11, wherein the dosage of methyl cinnamate ranges from 0.125% to 0.8%.

17. The method of claim 11, wherein methyl cinnamate is administered in oral, parenteral, subcutaneous, intramuscular, intravenous, transdermal or aerosol form.

18. The method of claim 11, wherein methyl cinnamate is contained in essential oil of Zanthoxylum armatum.

19. The method of claim 18, wherein the amount of essential oil of Zanthoxylum armatum ranges from 0.008% to 0.3%.

20. The method of claim 18, wherein the amount of essential oil of Zanthoxylum armatum ranges from 0.008% to 0.125%.

21. The method of claim 18, wherein the amount of essential oil of Zanthoxylum armatum ranges from 0.125% to 0.3%.
